(19) United States
(12) Patent Application Publication
(10) Pub. No.: US 2026/0141430 A1
(43) Pub. Date: May 21, 2026

(54) METHODS AND SYSTEMS FOR A PRESCRIPTION MEDICATION EXCHANGE

(71) Applicant: Pricemypills LLC, Allen, TX (US)

(72) Inventor: Kirat Shahiwala, Parker, TX (US)

(73) Assignee: A2Z Pharmacy Consultancy LLC, Allen, TX (US)

(21) Appl. No.: 18/952,591

(22) Filed: Nov. 19, 2024

(51) Int. Cl. G06Q 30/0601; G06Q 30/0283; G16H 20/10

(52) U.S. Cl. CPC G06Q 30/0611; G16H 20/10

(57) ABSTRACT

A system for providing a procurement of prescription medication quotes includes a network interface coupling the system to patient computing systems, a prescription provider computing system, and pharmacy computing systems. One or more data storage devices are configured to store prescription data of a medication prescription prescribed by a prescription provider to one or more patients. One or more tangible, non-transitory memories are configured to store computer-executable instructions that, when executed by one or more processors of the system, cause the system to: generate a prescription profile relative to the medication prescription and determine whether or not the medication prescription is eligible to be covered by an insurance plan of each of the one or more patients. Based on the determination, additional steps are carried out that include the generation of bidding quotes for the medication prescription and the sending of the bidding quotes to the one or more patients.

### BACKGROUND

Patients that are prescribed a prescription for medication are generally sensitive to the cost of their medication and often seek out the best prices for their medication based on their insurance coverage. This is especially true if the medication is a significant amount of the patient's monthly income. Due to the fact that there are so many different pharmacy companies, the process of 1) finding and 2) comparing medication prices may be overwhelming and time consuming for the patient. Additionally, there is a lack of awareness and support for local independent pharmacies who can significantly help patients in need of better cash-based discounts.

### BRIEF DESCRIPTION OF THE DRAWINGS

For a more complete understanding of the features and advantages of the present disclosure, reference is now made to the detailed description along with the accompanying figures in which corresponding numerals in the different figures refer to corresponding parts and in which:

FIG. 1 is an illustration of exemplary environment including components associated with providing a procurement of prescription medication quotes based on a medication prescription, in accordance with certain example embodiments;

FIG. 2 is an illustration of a computing machine and a system applications module, in accordance with certain example embodiments;

FIG. 3 is an illustration of a computer-implemented method for providing and procuring prescription medication quotes in accordance with certain example embodiments; and

FIG. 4, which is an illustration of an exemplary patient portal page for an Internet website for a prescription medication exchange system in accordance with certain example embodiments.

The illustrated figures are only exemplary and are not intended to assert or imply any limitation with regard to the environment, architecture, design, or process in which different examples may be implemented.

### DETAILED DESCRIPTION

The present invention relates generally to prescription medication and, more specifically, to systems and methods for providing a procurement of prescription medication quotes based on a medication prescription. The term “prescription provider” is used herein to describe a medical practitioner who is certified to prescribe a prescription medication and may be any of: a doctor, a pharmacist, a nurse practitioner, a physician assistant, or a clinical psychologist. The term “patient” is used herein to describe an individual who is prescribed a medication from any of: a doctor, a pharmacist, a nurse practitioner, a physician assistant, or a clinical psychologist.

The teachings of certain embodiments of the present invention allow the implementation of an improved system to aggregate offers for the price of specific medications from multiple prescription medication providers. By submitting a single medication prescription to the computing system disclosed, a patient can receive multiple offers, or “bids”, for the subject medication of the prescription without having to take the time or energy to seek out and compare offers for the medication, which significantly reduces network traffic and resources when compared to known methods and systems of medication procurement. Additionally, the system may help prescription medication providers, such as independent pharmacies, to minimize their monthly operating costs by combining different operational services and creating a “one-stop shop” for all of the providers'needs.

While the making and using of various embodiments of the present disclosure are discussed in detail below, it should be appreciated that the present disclosure provides many applicable inventive concepts, which can be embodied in a wide variety of specific contexts. The specific embodiments discussed herein are merely illustrative and do not delimit the scope of the present disclosure. In the interest of clarity, not all features of an actual implementation may be described in the present disclosure.

Unless otherwise indicated, all numbers expressing quantities of components and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the examples of the present invention. At the very least, and not as an attempt to limit the application of the doctrine of equivalents to the scope of the claim, each numerical parameter should at least be construed in light of the number of reported significant digits and by applying ordinary rounding techniques. It should be noted that when “about” is at the beginning of a numerical list, “about” modifies each number of the numerical list. Further, in some numerical listings of ranges some lower limits listed may be greater than some upper limits listed. One skilled in the art will recognize that the selected subset will require the selection of an upper limit in excess of the selected lower limit.

Presented herein is a system for providing a procurement of prescription medication quotes based on a medication prescription. The system comprises a network interface communicatively configured to couple the system to one or more patient computing systems of one or more patients, a prescription provider computing system, and a plurality of pharmacy computing systems. One or more data storage devices are configured to store prescription data relative to the medication prescription prescribed by a prescription provider to one or more patients. One or more tangible, non-transitory memories store computer-executable instructions that, when executed by one or more processors of the system, cause the system to generate a prescription profile based upon the stored prescription data relative to the medication prescription prescribed to the one or more patients and determine, based on the prescription profile, whether or not the medication prescription is eligible to be covered by an insurance plan of each of the one or more patients.

When the medication prescription is determined to not be eligible to be covered by at least one of the insurance plans of the one or more patients or is determined to be eligible to be covered by at least one of the insurance plans of the one or more patients but the medication prescription is associated with a copay above a threshold cost relative to the at least one of the insurance plans, the instructions may be executed to further generate bidding quotes for the medication prescription by providing, via the network interface, the prescription profile of the stored prescription data to the plurality of pharmacy computing systems. The instructions may be executed to further receive, from one or more of the plurality of pharmacy computing systems via the network interface, the bidding quotes for the medication prescription, wherein each of the plurality of bidding quotes is generated by one of the plurality of pharmacy computing systems based on the prescription profile. The instructions may be executed to further send one or more notifications to each of the one or more patient computing systems for review by respective patients of the one or more patients of the one or more patient computing systems via respective user interfaces of the one or more patient computing systems. The instructions may be executed to further receive, by the system, one or more notifications containing information relative to a selection of one of the bidding quotes for each of the one or more patients of the one or more patient computing systems. The instructions may be executed to further send, to respective pharmacy computing systems of the plurality of pharmacy computing systems associated with the one or more of the selected one of the bidding quotes via the network interface, one or more notifications containing information relative to the selection of the one of the bidding quotes.

Also presented herein is a computer-implemented method for providing a procurement of prescription medication quotes based on a medication prescription. The method comprises generating, by one or more processors of a first computing system, a prescription profile based on stored prescription data relative to a medication prescription prescribed to one or more patients. Once the prescription profile is generated, the medication prescription is, based on the prescription profile, determined to be eligible (or not) to be covered by an insurance plan of each of the one or more patients by the one or more processors of the first computing system. When the medication prescription is determined to not be eligible to be covered by at least one of the insurance plans of the one or more patients or is determined to be eligible to be covered by at least one of the insurance plans of the one or more patients but the medication prescription is associated with a copay above a threshold cost relative to the at least one of the insurance plans, additional steps are carried out.

The method further includes generating bidding quotes for the medication prescription by providing, via a network interface of the first computing system, the prescription profile of the stored prescription data to a plurality of pharmacy computing systems that are operated by a plurality of pharmacies. The bidding quotes for the medication prescription are then received at the first computing system from one or more of the plurality of pharmacy computing systems, where each of the plurality of bidding quotes is generated by one of the plurality of pharmacy computing systems based on the prescription profile. One or more notifications are then sent, via the network interface of the first computing system, to each of the one or more patient computing systems for review by respective patients of the one or more patients of the one or more patient computing systems via respective user interfaces of the one or more patient computing systems. One or more notifications containing information relative to a selection of one of the bidding quotes for each of the one or more patients of the one or more patient computing systems are then received by the network interface of the first computing system. Once the notifications are received by the first computing system, one or more notifications containing information relative to the selection of the one of the bidding quotes are sent to respective pharmacy computing systems of the plurality of pharmacy computing systems associated with the one or more of the selected one of the bidding quotes via the network interface of the first computing system.

Also presented herein is a non-transitory computer-readable storage medium storing one or more programs configured to be executed by one or more processors of a first computing system having a network interface, the one or more programs including instructions for carrying out a number of steps. The instructions include generating, by the one or more processors of the first computing system, a prescription profile based upon stored prescription data relative to a medication prescription prescribed to one or more patients. The instructions further include determining, by the one or more processors of the first computing system based on the prescription profile, whether or not the medication prescription is eligible to be covered by an insurance plan of each of the one or more patients. When the medication prescription is determined to not be eligible to be covered by at least one of the insurance plans of the one or more patients or is determined to be eligible to be covered by at least one of the insurance plans of the one or more patients but the medication prescription is associated with a copay above a threshold cost relative to the at least one of the insurance plans, additional instructions are implemented for carrying out a number of steps. The instructions further include generating bidding quotes for the medication prescription by providing, via the network interface of the first computing system, the prescription profile of the stored prescription data to a plurality of pharmacy computing systems that are operated by a plurality of pharmacies. The instructions further include receiving, from one or more of the plurality of pharmacy computing systems via the network interface, the bidding quotes for the medication prescription at the first computing system, wherein each of the plurality of bidding quotes is generated by one of the plurality of pharmacy computing systems based on the prescription profile. The instructions further include sending, via the network interface of the first computing system, one or more notifications to each of the one or more patient computing systems for review by respective patients of the one or more patients of the one or more patient computing systems via respective user interfaces of the one or more patient computing systems. The instructions further include receiving, by the network interface of the first computing system, one or more notifications containing information relative to a selection of one of the bidding quotes for each of the one or more patients of the one or more patient computing systems. The instructions further include sending, to respective pharmacy computing systems of the plurality of pharmacy computing systems associated with the one or more of the selected one of the bidding quotes via the network interface of the first computing system, one or more notifications containing information relative to the selection of the one of the bidding quotes.

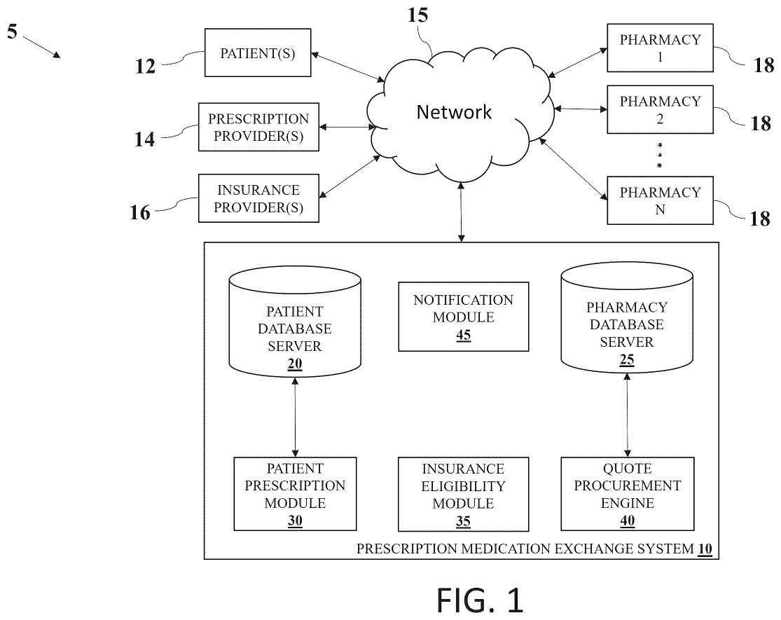

FIG. 1 is an illustration of exemplary environment 5 including components associated with providing a procurement of prescription medication quotes based on a medication prescription, in accordance with certain example embodiments. As shown in FIG. 1, the environment 5 may include a computing device 12 that is operated by a patient of a prescription provider. Computing device 12 may be any suitable type of computing device having wired and/or wireless communication capabilities, such as a personal computer, tablet, phablet, smartphone, etc.

Environment 5 may include a prescription medication exchange system 10, which may comprise a computing system. Prescription medication exchange system 10 may include one or more servers, or may include a plurality of networked computing devices that have an appearance of a single, logical computing device or system, e.g., a group of cloud computing devices, a peer group of computing devices, or the like. Prescription medication exchange system 14 may be communicatively coupled to computing device 12 via one or more electronic or communications networks. The one or more electronic or communications networks may be a single network (as shown, network 15), or may include multiple networks of one or more types (e.g., one or more wired and/or wireless local area networks (LANs), and/or one or more wired and/or wireless wide area networks (WANs) such as the Internet), for example.

Environment 5 may include computing systems 18 associated with N respective pharmacies (e.g., three pharmacies, seven pharmacies, etc.). Each of the computing systems 18 may include one or more servers of the respective pharmacy, or may include a plurality of networked computing devices that have an appearance of a single, logical computing device or system, e.g., a group of cloud computing devices, a peer group of computing devices, or the like. Each of the pharmacy computing systems 18 may be communicatively coupled to computing system 12, computing system 14, computing system 16, and/or prescription medication exchange system 10 via one or more electronic or communications networks (as shown, network 15). Each of the pharmacies 18 may be a company or organization providing particular types of pharmaceutical medications that require a medication prescription prescribed by a prescription provider, such as a doctor, for example.

Prescription medication exchange system 10 may include various components, including a patient prescription module 30, an insurance eligibility module 35, a quote procurement engine 40, and/or a notification module 45. Each of some or all of the components 30, 35, 40, 45 may be (or may include) a respective set of one or more computing devices or processors that executes software or computer-executable instructions to perform the corresponding functions described herein. Alternatively, each of some or all of the components 30, 35, 40, and 45 may be, or include, a respective component of software or set of computer-executable instructions that is stored on one or more tangible, non-transitory computer-readable media (e.g., a random access memory (RAM) and/or read-only memory (ROM) of the prescription medication exchange system 10) and that is executed by one or more processors of the prescription medication exchange system 10 to perform the corresponding functions described herein. Still additionally or alternatively, each of some or all of the components 30, 35, 40, and 45 may at least partially comprise hardware and/or firmware. Further, one or more of the components 30, 35, 40 and 45 may be combined into a single unit, or may be omitted. In various different embodiments, for example, the prescription medication exchange system 10 may not include any one of components 30, 35, 40, and/or 45.

In one embodiment, patient prescription module 30 may generate a prescription profile for one or more patients 12 based on stored prescription data relative to the medication prescription prescribed to each patient 12. The stored prescription data may be received at prescription medication exchange system 10 from system 14 of the prescription provider (such as, for example, a doctor, a pharmacist, a nurse practitioner, a physician assistant, or a clinical psychologist) of the one or more patients 12, and the received results may be stored in a patient database server 20, which may be configured to store the stored prescription data of multiple patients. Patient database server 20 may be any suitable type of persistent memory, and may comprise one or more data storage devices that have the appearance of a single, logical data storage device. Additionally or alternatively, patient prescription module 30 may be triggered to generate a prescription profile for one or more patients 12 when corresponding prescription data is stored in patient database server 20. For example, the act of initially storing new prescription data may trigger patient prescription module 30 to generate a corresponding prescription profile. Additionally or alternatively, patient prescription module 30 may regularly scan patient database server 20 for newly added prescription data and/or generate new prescription profiles therefrom, and/or patient prescription module 30 may generate prescription profiles when directed to by a user or by another system.

Patient prescription module 30 may utilize the obtained prescription data to generate a prescription profile for one or more patients 12. Generally speaking, the prescription data of the one or more patients 12 may include identifying information or analysis results relative to the one or more patients 12, or may be any other data or information that was obtained from a prescription provider 14. In an embodiment, a prescription profile may include information including at least one of: a number of patients 12 that the prescription medication is prescribed to, a prescription medication name, a prescription medication strength, a prescription medication quantity, a prescription medication identification number, prescription medication instructions, insurance provider information, and/or refill information. It is noted that a prescription profile may include information/data that has been pre-determined to be important in determining a price of a prescription medication, and a pharmacy 18 may utilize the prescription profile to determine a price of (or a bid for) a prescription medication of a prescription profile. In an embodiment, a “price” may refer to the cost of a single monthly payment of a prescription medication.

Insurance eligibility module 35 of prescription medication exchange system 10 may determine, based on the contents of a prescription profile, whether or not the corresponding prescription medication is eligible to be covered by insurance or not. The determination may be based upon, for example, insurance eligibility information data (criteria) provided to insurance eligibility module 35 via a computing system of insurance provider 16 (the insurance/insurance eligibility information data relative to a patient 12). Additionally, insurance eligibility module 35 may determine a deductible and/or copay for a prescription medication. This determination/copay may be based upon, for example, deductible/copay information data (criteria) provided to insurance eligibility module 35 via a computing system of insurance provider 16. It is noted that in the case where the prescription profile is associated with more than one patient 12, each of the patients 12 may have the same insurance provider.

Information, data, and/or other criteria that are provided by patient(s) 12 and/or by prescription provider(s) 14 and/or by insurance provider(s) 16 and/or by pharmacies 18 may be stored in patient database server 20, in one embodiment, and may be accessed by patient prescription module 30 and/or by insurance eligibility module 35 and/or by quote procurement engine 40. Patient database server 20 may be any suitable type of persistent memory, and may comprise one or more data storage devices that have the appearance of a single, logical data storage device, although in some cases, patient database server 20 and pharmacy database server 25 may be an integral database.

Quote procurement engine 40 of prescription medication exchange system 10 may generate a procurement of bidding quotes for one or more patients 12 based on a corresponding prescription profile. The bidding quotes may be received at prescription medication exchange system 10 from system(s) 18 of one or more pharmacies, and the received bidding quotes may be stored in a pharmacy database server 25, which may be configured to store the bidding quotes. Pharmacy database server 25 may be any suitable type of persistent memory, and may comprise one or more data storage devices that have the appearance of a single, logical data storage device. Additionally or alternatively, quote procurement engine 40 may be triggered to generate a procurement of bidding quotes for one or more patients 12 once corresponding bidding quote data is stored in pharmacy database server 25. For example, the act of initially storing new bidding quote data may trigger quote procurement engine 40 to generate a procurement of bidding quotes for a corresponding prescription profile. Additionally or alternatively, quote procurement engine 40 may regularly scan pharmacy database server 25 for newly added bidding quote data to add to a generated procurement of bidding quotes (as a result of one or more pharmacies 18 sending bidding quote data to pharmacy database server 25 at different times).

Information, data, and/or other criteria relative to bidding quotes on prescription medications provided by pharmacies 18 may be stored in pharmacy database server 25 or in another database.

In embodiments, prescription medication exchange system 10 may receive the bidding quotes from the pharmacy computing systems 18 and may cause multiple bidding quotes to be presented to one or more patients of patient computing systems 12 via notification module 45. In additional embodiments, notification module 45 may perform at least one of: providing a prescription profile of stored prescription data to a plurality of pharmacy computing systems 18, sending one or more notifications to each of the one or more patient computing systems 12 for review by respective patients of the one or more patient computing systems 12, and sending, to respective pharmacy computing systems 18 of the bidding quotes, one or more notifications containing information relative to the selection of the bidding quotes.

In embodiments, stored prescription data relative to a medication prescription for a single patient 12 may be provided to prescription medication exchange system 10 by either of a prescription provider 14 or a patient 12 via respective prescription provider computing system 14 or patient computing system 12. In the case where a single medication prescription is associated with multiple patients 12, for ease of information exchange, a prescription provider 14 may provide stored prescription data for multiple patients 12 to prescription medication exchange system 10 via prescription provider computing system 14.

In embodiments, one or more pharmacies of pharmacy computing systems 18 may utilize prescription medication exchange system 10 to negotiate bidding quotes for prescription medication with an insurance provider 16 of insurance provider computing systems 16. In this instance, a pharmacy 18 may create its own prescription profile (pharmacy prescription profile) that may be sent to prescription medication exchange system 10 and subsequently sent to an insurance provider 16 of insurance provider computing systems 16. The pharmacy prescription profile may include a proposal to offer a certain prescription medication at a certain price and quantity. If the pharmacy prescription profile is approved by the insurance provider, the proposal may be converted into bidding quotes that may be offered in response to a pharmacy 18 receiving a prescription profile of a patient 12. In certain instances, the prescription medication may be offered at a loss to the pharmacy 18 (as bidding quotes), which can lead to a pharmacy 18 to acquire additional customers/patients 12. For example, for patients 12 that eventually choose this bidding quote of the pharmacy 18 associated with the pharmacy prescription profile, the patient 12 may be more likely to consider choosing that pharmacy 18 for any other prescription medications that the patient 12 may have.

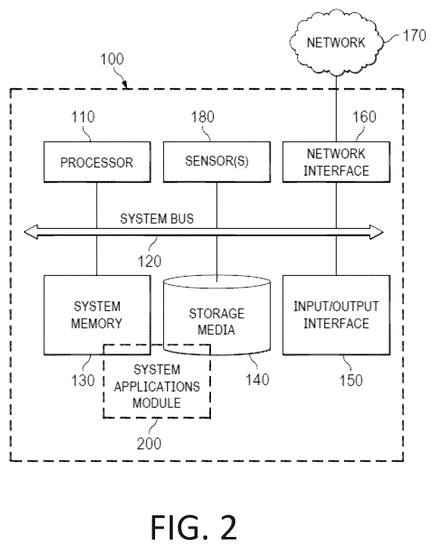

Referring now to FIG. 2, illustrated is a computing machine 100 and a system applications module 200, in accordance with example embodiments. The computing machine 100 can correspond to any of the various computers, mobile devices, laptop computers, Internet of Things (IOT), servers, embedded systems, or computing systems presented herein. The module 200 can comprise one or more hardware or software elements, e.g. other OS application and user and kernel space applications, designed to facilitate the computing machine 100 in performing the various methods and processing functions presented herein. The computing machine 100 can include various internal or attached components such as a processor 110, system bus 120, system memory 130, storage media 140, input/output interface 150, a network interface 160 for communicating with a network 170, e.g. cellular/GPS, Bluetooth, WIFI, or Devicenet, EtherCAT, Analog, RS485, etc., and one or more sensors 180.

The computing machines 100 can be implemented as a conventional computer system, an embedded controller, a laptop, a server, a mobile device, a smartphone, a wearable computer, a customized machine, any other hardware platform, or any combination or multiplicity thereof. The computing machines 100 can be a distributed system configured to function using multiple computing machines interconnected via a data network or bus system.

Processor 110 can be designed to execute code instructions in order to perform the operations and functionality described herein, manage request flow and address mappings, and to perform calculations and generate commands. Processor 110 can be configured to monitor and control the operation of the components in the computing machines 100. Processor 110 can be a general purpose processor, a processor core, a multiprocessor, a reconfigurable processor, a microcontroller, a digital signal processor (“DSP”), an application specific integrated circuit (“ASIC”), a controller, a state machine, gated logic, discrete hardware components, any other processing unit, or any combination or multiplicity thereof. Processor 110 can be a single processing unit, multiple processing units, a single processing core, multiple processing cores, special purpose processing cores, co-processors, or any combination thereof. According to certain embodiments, processor 110 along with other components of computing machine 100 can be a software based or hardware based virtualized computing machine executing within one or more other computing machines.

The system memory 130 can include non-volatile memories such as read-only memory (“ROM”), programmable read-only memory (“PROM”), erasable programmable read-only memory (“EPROM”), flash memory, or any other device capable of storing program instructions or data with or without applied power. The system memory 130 can also include volatile memories such as random access memory (“RAM”), static random access memory (“SRAM”), dynamic random access memory (“DRAM”), and synchronous dynamic random access memory (“SDRAM”). Other types of RAM also can be used to implement the system memory 130. The system memory 130 can be implemented using a single memory module or multiple memory modules. While the system memory 130 is depicted as being part of the computing machine 100, one skilled in the art will recognize that the system memory 130 can be separate from the computing machine 100 without departing from the scope of the subject technology. It should also be appreciated that the system memory 130 can include, or operate in conjunction with, a non-volatile storage device such as the storage media 140.

The storage media 140 can include a hard disk, a floppy disk, a compact disc read-only memory (“CD-ROM”), a digital versatile disc (“DVD”), a Blu-ray disc, a magnetic tape, a flash memory, other non-volatile memory device, a solid state drive (“SSD”), any magnetic storage device, any optical storage device, any electrical storage device, any semiconductor storage device, any physical-based storage device, any other data storage device, or any combination or multiplicity thereof. The storage media 140 can store one or more operating systems, application programs and program modules, data, or any other information. The storage media 140 can be part of, or connected to, the computing machine 100. The storage media 140 can also be part of one or more other computing machines that are in communication with the computing machine 100 such as servers, database servers, cloud storage, network attached storage, and so forth.

The applications module 200 and other OS application modules can comprise one or more hardware or software elements configured to facilitate the computing machine 100 with performing the various methods and processing functions presented herein. The applications module 200 and other OS application modules can include one or more algorithms or sequences of instructions stored as software or firmware in association with the system memory 130, the storage media 140 or both. The storage media 140 can therefore represent examples of machine or computer readable media on which instructions or code can be stored for execution by the processor 110. Machine or computer readable media can generally refer to any medium or media used to provide instructions to the processor 110. Such machine or computer readable media associated with the applications module 200 and other OS application modules can comprise a computer software product. It should be appreciated that a computer software product comprising the applications module 200 and other OS application modules can also be associated with one or more processes or methods for delivering the applications module 200 and other OS application modules to the computing machine via a network, any signal-bearing medium, or any other communication or delivery technology. The applications module 200 and other OS application modules can also comprise hardware circuits or information for configuring hardware circuits such as microcode or configuration information for an FPGA or other PLD. In one exemplary embodiment, applications module 200 and other OS application modules can include algorithms capable of performing the functional operations described by the flow charts (modes of operation) computer systems presented herein.

The input/output (“I/O”) interface 150 can be configured to couple to one or more external devices, to receive data from the one or more external devices, and to send data to the one or more external devices. Such external devices along with the various internal devices can also be known as peripheral devices. The I/O interface 150 can include both electrical and physical connections for coupling the various peripheral devices to the computing machine 100 or the processor 110. The I/O interface 150 can be configured to communicate data, addresses, and control signals between the peripheral devices, the computing machine 100, or the processor 110. The I/O interface 150 can be configured to implement any standard interface, such as small computer system interface (“SCSI”), serial-attached SCSI (“SAS”), fiber channel, peripheral component interconnect (“PCI”), PCI express (PCIe), serial bus, parallel bus, advanced technology attached (“ATA”), serial ATA (“SATA”), universal serial bus (“USB”), Thunderbolt, FireWire, various video buses, and the like. The I/O interface 150 can be configured to implement only one interface or bus technology. Alternatively, the I/O interface 150 can be configured to implement multiple interfaces or bus technologies. The I/O interface 150 can be configured as part of, all of, or to operate in conjunction with, the system bus 120. The I/O interface 150 can include one or more buffers for buffering transmissions between one or more external devices, internal devices, the computing machine 100, or the processor 110.

The I/O interface 150 can couple the computing machine 100 to various input devices including mice, touch-screens, scanners, electronic digitizers, sensors, receivers, touchpads, trackballs, cameras, microphones, keyboards, any other pointing devices, or any combinations thereof. The I/O interface 150 can couple the computing machine 100 to various output devices including video displays, speakers, printers, projectors, tactile feedback devices, automation control, robotic components, actuators, motors, fans, solenoids, valves, pumps, transmitters, signal emitters, lights, and so forth.

The computing machine 100 can operate in a networked environment using logical connections through the NIC 160 to one or more other systems or computing machines across a network 170. The network 170 can include wide area networks (WAN), local area networks (LAN), intranets, the Internet, wireless access networks, wired networks, mobile networks, telephone networks, optical networks, or combinations thereof. The network 170 can be packet switched, circuit switched, of any topology, and can use any communication protocol. Communication links within the network 170 can involve various digital or an analog communication media such as fiber optic cables, free-space optics, waveguides, electrical conductors, wireless links, antennas, radio-frequency communications, and so forth.

The one or more sensors 180 can be a position sensor. The position sensor can be a capacitive, optical, strain gauge, or magnetic sensor. The sensors 180 can be traditional sensors or semiconductor based sensors.

The processor 110 can be connected to the other elements of the computing machine 100 or the various peripherals discussed herein through the system bus 120. It should be appreciated that the system bus 120 can be within the processor 110, outside the processor 110, or both. According to some embodiments, any of the processors 110, the other elements of the computing machine 100, or the various peripherals discussed herein can be integrated into a single device such as a system on chip (“SOC”), system on package (“SOP”), or ASIC device.

Embodiments may comprise a computer program that embodies the functions described and illustrated herein, wherein the computer program is implemented in a computer system that comprises instructions stored in a machine-readable medium and a processor that executes the instructions. However, it should be apparent that there could be many different ways of implementing embodiments in computer programming, and the embodiments should not be construed as limited to any one set of computer program instructions unless otherwise disclosed for an exemplary embodiment. Further, a skilled programmer would be able to write such a computer program to implement an embodiment of the disclosed embodiments based on the appended flow charts, algorithms and associated description in the application text. Therefore, disclosure of a particular set of program code instructions is not considered necessary for an adequate understanding of how to make and use embodiments. Further, those skilled in the art will appreciate that one or more aspects of embodiments described herein may be performed by hardware, software, or a combination thereof, as may be embodied in one or more computing systems. Moreover, any reference to an act being performed by a computer should not be construed as being performed by a single computer as more than one computer may perform the act.

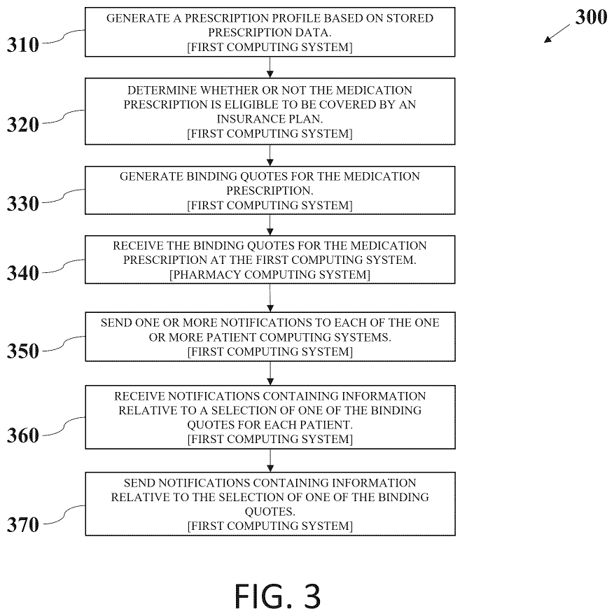

FIG. 3 is an illustration of a computer-implemented method 300 for providing and procuring prescription medication quotes in accordance with certain example embodiments. In an embodiment, at least a portion of method 300 may be performed by prescription medication exchange system 10 of FIG. 1 and/or by computing machine 100 of FIG. 2. For example, method 300 may be performed by executing particular computer-executable instructions stored on prescription medication exchange system 10. Indeed, method 300 may operate in conjunction with environment 5, prescription medication exchange system 10 illustrated in FIG. 1, computing machine 100 of FIG. 2, and/or any one or more portions of any one or more of the other methods described herein. It is noted that environment 5 may include prescription medication exchange system 10 communicatively coupled to one or more patient computing systems 12, a prescription provider computing system 14, an insurance provider computing system 16, and a plurality of pharmacy computing systems 18 via network 15. It is further noted that first computing system (as presented below) may refer to either of prescription medication exchange system 10 or computing machine 100.

Method 300 may include generating, by one or more processors of a first computing system (such as, for example, prescription medication exchange system 10 or computing machine 100), a prescription profile based on stored prescription data relative to a medication prescription prescribed to one or more patients 12 (Block 310). The medication prescription may, for example be uploaded to prescription provider computing system 14 and sent to prescription medication exchange system 10 via network 15. Once the prescription profile is generated, the one or more processors of the first computing system determines, based on the prescription profile, whether or not the medication prescription is eligible to be covered by an insurance plan of each of the one or more patients 12 (Block 320).

When the medication prescription is determined to not be eligible to be covered by at least one of the insurance plans of the one or more patients or is determined to be eligible to be covered by at least one of the insurance plans of the one or more patients 12 but the medication prescription is associated with a copay above a threshold cost relative to the at least one of the insurance plans, a number of additional steps of method 300 may be carried out. Method 300 further includes generating bidding quotes for the medication prescription by providing, via a network interface of the first computing system, the prescription profile of the stored prescription data to a plurality of pharmacy computing systems 18 that are operated by a plurality of pharmacies 18 (Block 330). In an additional embodiment, the additional steps of method 300 may be carried out only once the medication prescription is determined to not be eligible to be covered by at least one of the insurance plans of the one or more patients 12.

The bidding quotes for the medication prescription are then received at the first computing system from one or more of the plurality of pharmacy computing systems 18 via the network interface, where each of the plurality of bidding quotes is generated by one of the plurality of pharmacy computing systems 18 based on the prescription profile (Block 340). Once the bidding quotes are received, one or more notifications are sent, via the network interface of the first computing system, to each of the one or more patient computing systems 12 for review by respective patients 12 of the one or more patients 12 of the one or more patient computing systems 12 via respective user interfaces of the one or more patient computing systems 12 (Block 350). One or more notifications containing information relative to a selection of one of the bidding quotes for each of the one or more patients 12 of the one or more patient computing systems 12 are then received by the network interface of the first computing system (Block 360). Once the one or more notifications are received by the network interface of the first computing system, one or more notifications containing information relative to the selection of the one of the bidding quotes are sent to respective pharmacy computing systems 18 of the plurality of pharmacy computing systems 18 associated with the one or more of the selected one of the bidding quotes via the network interface of the first computing system (Block 370).

In an embodiment, the prescription provider 14 is one of: a doctor, a pharmacist, a nurse practitioner, a physician assistant, or a clinical psychologist.

In an embodiment, the prescription profile comprises a number of the one or more patients 12 that are prescribed the medication prescription.

In an embodiment, each of the one or more notifications containing the information relative to the selection of the one of the bidding quotes comprises identifying information of the one or more patients 12 associated with each of the one or more notifications containing the information relative to the selection of the one of the bidding quotes.

In an embodiment, method 300 further comprises sending, to the one or more patient computing systems 12 of the plurality of patient computing systems 12 associated with the one or more notifications containing the information relative to the selection of the one of the bidding quotes via the network interface of the first computing system, where one or more notifications containing identifying information of the respective pharmacy computing systems 18 of the plurality of pharmacy computing systems 18 are associated with the one or more of the selected one of the bidding quotes.

In an embodiment, the generating of the bidding quotes for the medication prescription may be performed at a scheduled interval, where the scheduled interval is communicated to the first computing system from a respective one of the one or more patients 12 via the one or more patient computing systems 12.

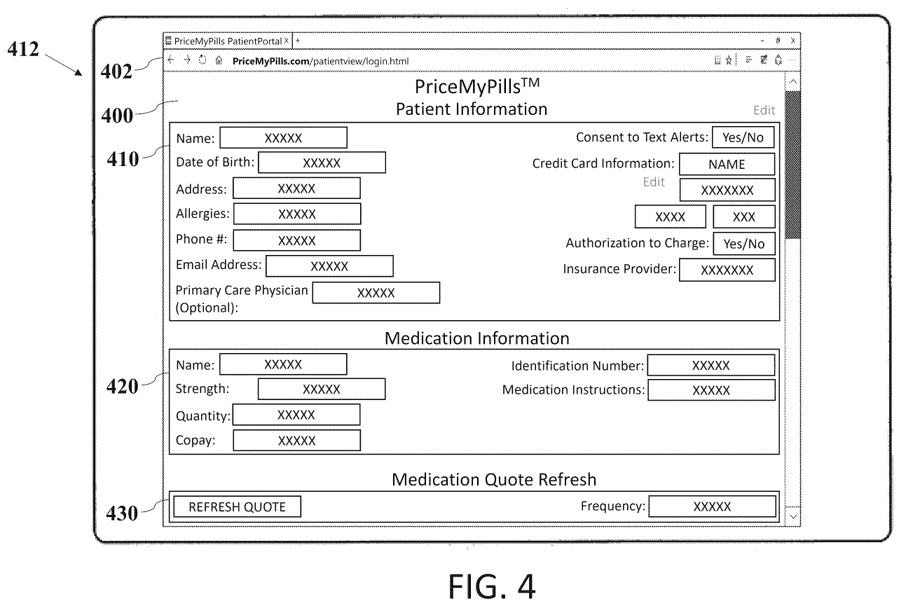

FIG. 4 is an illustration of an exemplary patient portal page 400 for an Internet website for a prescription medication exchange system 10 in accordance with certain example embodiments. As shown, patient portal page 400 is presented within a user interface 402 of an exemplary patient computing system 412 (in this instance, a tablet). Patient portal page 400 includes a patient information section 410, a medication information section 420, and a medication quote refresh section 430. Patient information section 410 includes a plurality of engageable icons with which a patient may input identifying information relative to the patient 12. Medication information section 420 includes a plurality of engageable icons with which a patient 12 may input information relative to a prescription medication of the patient 12. It is noted that some or all of the information input into one or both of patient information section 410 or medication information section 420 may be included in a prescription profile associated with a patient computing system 12. As further shown, medication quote refresh section 430 includes a “REFRESH QUOTE” engageable icon and a “Frequency” engageable icon. These icons may be utilized in relation to the generating of a bidding quote for a medication prescription that is performed at a scheduled interval. Prior to a patient engaging the “REFRESH QUOTE” engageable icon, the patient 12 may input how frequently they would like to receive a bidding quote for a specific prescription medication (such as, for example: weekly, every two weeks, monthly, etc.). Once the frequency is input, the patient 12 may engage the “REFRESH QUOTE” engageable icon, which may send a request to prescription medication exchange system 10 indicating to send to the patient 12 refreshed bidding quotes for the associated prescription medication from one or more pharmacies 18 (based on the input frequency).

In embodiments, in relation to the generating of bidding quotes for a medication prescription, each of the pharmacies 18 of pharmacy computing systems 18 may, after negotiating with an insurance provider 16, offer a bidding quote that is the same price as a price presented in a prescription profile, less than a price presented in a prescription profile, or the same as or less than a price presented in a prescription profile and including an additional product or service. The additional products or services may be included in a prescription profile and may include at least one of: free home delivery, same day fulfilment, free products (such as a pill pack, for example), nutritional supplements, or discounts on any of the aforementioned. In this instance, the pharmacy 18 may offer these bidding quotes to patients 12 in order to attempt to boost overall volume of accepted bidding quotes.

In embodiments, a pharmacy 18 may, after negotiating with an insurance provider 16, provide a bidding quote to a patient 12, where the bidding quote is below a market value of the prescription medication that is the subject of the bidding quote. In this instance, the pharmacy 18 may offer the bidding quote to patients 12 that are considered more likely to have a tendency to, for example, buy higher margin prescription medication or have multiple prescription medications. This may eventually lead to additional accepted bidding quotes for the pharmacy 18.

In embodiments, a pharmacy 18 may, after negotiating with an insurance provider 16, provide a plurality of bidding quotes to a plurality of patients 12, where the bidding quotes are either offered at a loss to the pharmacy 18 or are offered with a rebate (which may also cause a loss to the pharmacy 18). In this instance, the pharmacy 18 may offer these bidding quotes to patients 12 in order to attempt to boost overall volume of accepted bidding quotes.

In embodiments, prescription medication exchange system 10 may restrict one or more pharmacies 18 from providing bidding quotes to certain patients 12 based on a demographics filter implemented by prescription medication exchange system 10. The demographics filter may restrict pharmacies 18 from providing bidding quotes based on at least one of: gender, age, income location, employment, occupation, and living status.

In embodiments, in relation to a medication prescription where bidding quotes have been generated, each of the pharmacy computing systems 18 of pharmacies that are offering competing bidding quotes for the medication prescription may be sent a notification from prescription medication exchange system 10 that includes the competing bidding quotes of the other pharmacies 18. Each pharmacy 18, in response to receiving the notification including the competing bidding quotes, may amend their bidding quote and resubmit the amended bidding quote to prescription medication exchange system 10 for eventual review by one or more patients 12 via one or more patient computing systems 12.

In embodiments, the service of offering bidding quotes to patients 12 by pharmacies 18 via prescription medication exchange system 10 may be a paid service paid by the patients 12 receiving the bidding quotes as well as the pharmacies 18 offering the bidding quotes. In other embodiments, the service may be a paid service paid by only the pharmacies 18 offering the bidding quotes.

In embodiments, bidding quotes from pharmacies 18 for a single patient 12 may be aggregated by prescription medication exchange system 10 and sent to the patient 12 in a specific order based on one or more criteria. The criteria may include, but is not limited to: proximity of a pharmacy 18 to the patient 12, whether a pharmacy 18 is paying for a service (as described above; can be based on a tiered, paid system), or ratings of a pharmacy 18 by one or more patients 12. In specific embodiments, a pharmacy 18 may be assigned an HTR (High Transfer Rating), which may provide the pharmacy 18 the opportunity to present their bidding quotes to a patient 12 before other pharmacies 18. In order for a pharmacy 18 to receive an HTR rating, one or more patients 12 who have accepted a bidding quote from the pharmacy 18 may each send a notification to prescription medication exchange system 10 that each include: a rating based on only the price of a prescription medication associated with the accepted bidding quote and a rating for customer service of the pharmacy 18. A pharmacy's HTR rating may be an aggregated rating of the ratings presented in the above-mentioned notifications. It is noted that these notifications that include the HTR ratings from each patient 12 may be sent at any time after the patient 12 accepted a bidding quote from the pharmacy 18 (and typically after customer service has been rendered by the pharmacy 18).

In embodiments, the first computing system may refer to either of prescription medication exchange system 10 or computing machine 100. In additional embodiments, the first computing system may be operated and/or provided by one or more prescription provider(s) 14 and/or one or more insurance provider(s) 16 and/or one or more pharmacies 18 and/or one or more third parties (to the prescription provider(s) 14, insurance provider(s) 16, and/or pharmacies 18).

In embodiments, a pharmacy computing system 18 may include a prescription profile analysis engine (not depicted) that may be configured to analyze a prescription profile. As a result of analyzing the prescription profile, the prescription profile analysis engine may be configured to automatically send to prescription medication exchange system 10 a bidding quote that falls within a price range negotiated between the pharmacy 18 and an insurance provider 16.

In embodiments, one or more pharmacy computing systems 18 may include an automated intelligence engine that may assist a pharmacy 18 in providing bidding quotes to patients 12 in regard to health profiles or behaviors of the patients 12.

In embodiments, once a patient 12 has accepted a particular bidding quote from a particular pharmacy 18, identifying information of the patient 12 may be shared with the pharmacy 18 and identifying information of the pharmacy 18 may be shared with the patient 12.

It is noted that the prescription medications disclosed may include either of: generic prescription medications or brand-name prescription medications.

The example embodiments described herein can be used with computer hardware and software that perform the methods and processing functions described previously. The systems, methods, and procedures described herein can be embodied in a programmable computer, computer-executable software, or digital circuitry. The software can be stored on computer-readable media. For example, computer-readable media can include a floppy disk, RAM, ROM, hard disk, removable media, flash memory, memory stick, optical media, magneto-optical media, CD-ROM, etc. Digital circuitry can include integrated circuits, gate arrays, building block logic, field programmable gate arrays (FPGA), etc.

The example systems, methods, and acts described in the embodiments presented previously are illustrative, and, in alternative embodiments, certain acts can be performed in a different order, in parallel with one another, omitted entirely, and/or combined between different example embodiments, and/or certain additional acts can be performed, without departing from the scope and spirit of various embodiments. Accordingly, such alternative embodiments are included in the description herein.

As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

As used herein, “hardware” can include a combination of discrete components, an integrated circuit, an application-specific integrated circuit, a field programmable gate array, or other suitable hardware. As used herein, “software” can include one or more objects, agents, threads, lines of code, subroutines, separate software applications, two or more lines of code or other suitable software structures operating in two or more software applications, on one or more processors (where a processor includes one or more microcomputers or other suitable data processing units, memory devices, input-output devices, displays, data input devices such as a keyboard or a mouse, peripherals such as printers and speakers, associated drivers, control cards, power sources, network devices, docking station devices, or other suitable devices operating under control of software systems in conjunction with the processor or other devices), or other suitable software structures. In one exemplary embodiment, software can include one or more lines of code or other suitable software structures operating in a general purpose software application, such as an operating system, and one or more lines of code or other suitable software structures operating in a specific purpose software application. As used herein, the term “couple” and its cognate terms, such as “couples” and “coupled,” can include a physical connection (such as a copper conductor), a virtual connection (such as through randomly assigned memory locations of a data memory device), a logical connection (such as through logical gates of a semiconducting device), other suitable connections, or a suitable combination of such connections. The term “data” can refer to a suitable structure for using, conveying or storing data, such as a data field, a data buffer, a data message having the data value and sender/receiver address data, a control message having the data value and one or more operators that cause the receiving system or component to perform a function using the data, or other suitable hardware or software components for the electronic processing of data.

In general, a software system is a system that operates on a processor to perform predetermined functions in response to predetermined data fields. For example, a system can be defined by the function it performs and the data fields that it performs the function on. As used herein, a NAME system, where NAME is typically the name of the general function that is performed by the system, refers to a software system that is configured to operate on a processor and to perform the disclosed function on the disclosed data fields. Unless a specific algorithm is disclosed, then any suitable algorithm that would be known to one of skill in the art for performing the function using the associated data fields is contemplated as falling within the scope of the disclosure. For example, a message system that generates a message that includes a sender address field, a recipient address field and a message field would encompass software operating on a processor that can obtain the sender address field, recipient address field and message field from a suitable system or device of the processor, such as a buffer device or buffer system, can assemble the sender address field, recipient address field and message field into a suitable electronic message format (such as an electronic mail message, a TCP/IP message or any other suitable message format that has a sender address field, a recipient address field and message field), and can transmit the electronic message using electronic messaging systems and devices of the processor over a communications medium, such as a network. One of ordinary skill in the art would be able to provide the specific coding for a specific application based on the foregoing disclosure, which is intended to set forth exemplary embodiments of the present disclosure, and not to provide a tutorial for someone having less than ordinary skill in the art, such as someone who is unfamiliar with programming or processors in a suitable programming language. A specific algorithm for performing a function can be provided in a flow chart form or in other suitable formats, where the data fields and associated functions can be set forth in an exemplary order of operations, where the order can be rearranged as suitable and is not intended to be limiting unless explicitly stated to be limiting.

The above-disclosed embodiments have been presented for purposes of illustration and to enable one of ordinary skill in the art to practice the disclosure, but the disclosure is not intended to be exhaustive or limited to the forms disclosed. Many insubstantial modifications and variations will be apparent to those of ordinary skill in the art without departing from the scope and spirit of the disclosure. The scope of the claims is intended to broadly cover the disclosed embodiments and any such modification. Further, the following clauses represent additional embodiments of the disclosure and should be considered within the scope of the disclosure:
Clause 1, a system for providing a procurement of prescription medication quotes based on a medication prescription, comprising: a network interface communicatively configured to couple the system to one or more patient computing systems of one or more patients, a prescription provider computing system, and a plurality of pharmacy computing systems; one or more data storage devices configured to store prescription data relative to the medication prescription prescribed by a prescription provider to one or more patients; and one or more tangible, non-transitory memories storing computer-executable instructions that, when executed by one or more processors of the system, cause the system to: generate a prescription profile based upon the stored prescription data relative to the medication prescription prescribed to the one or more patients; determine, based on the prescription profile, whether or not the medication prescription is eligible to be covered by an insurance plan of each of the one or more patients; and when the medication prescription is determined to not be eligible to be covered by at least one of the insurance plans of the one or more patients or is determined to be eligible to be covered by at least one of the insurance plans of the one or more patients but the medication prescription is associated with a copay above a threshold cost relative to the at least one of the insurance plans: generate bidding quotes for the medication prescription by providing, via the network interface, the prescription profile of the stored prescription data to the plurality of pharmacy computing systems; receive, from one or more of the plurality of pharmacy computing systems via the network interface, the bidding quotes for the medication prescription at the system, wherein each of the plurality of bidding quotes is generated by one of the plurality of pharmacy computing systems based on the prescription profile; send one or more notifications to each of the one or more patient computing systems for review by respective patients of the one or more patients of the one or more patient computing systems via respective user interfaces of the one or more patient computing systems; receive, by the system, one or more notifications containing information relative to a selection of one of the bidding quotes for each of the one or more patients of the one or more patient computing systems; and send, to respective pharmacy computing systems of the plurality of pharmacy computing systems associated with the one or more of the selected one of the bidding quotes via the network interface, one or more notifications containing information relative to the selection of the one of the bidding quotes.
Clause 2, the system of Clause 1, wherein the prescription provider is one of: a doctor, a pharmacist, a nurse practitioner, a physician assistant, or a clinical psychologist.
Clause 3, the system of Clause 1, wherein the prescription profile comprises a number of the one or more patients that are prescribed the medication prescription.
Clause 4, the system of Clause 1, wherein each of the one or more notifications containing the information relative to the selection of the one of the bidding quotes comprises identifying information of the one or more patients associated with each of the one or more notifications containing the information relative to the selection of the one of the bidding quotes.
Clause 5, the system of Clause 4, wherein the computer-executable instructions are further executable to: send, to the one or more patient computing systems of the plurality of patient computing systems associated with the one or more notifications containing the information relative to the selection of the one of the bidding quotes, one or more notifications containing identifying information of the respective pharmacy computing systems of the plurality of pharmacy computing systems associated with the one or more of the selected one of the bidding quotes.
Clause 6, the system of Clause 1, wherein the generating of the bidding quotes for the medication prescription is performed at a scheduled interval, further wherein the scheduled interval is communicated to the system from a respective one of the one or more patients via the one or more patient computing systems.
Clause 7, a computer-implemented method for providing a procurement of prescription medication quotes based on a medication prescription, comprising: generating, by one or more processors of a first computing system, a prescription profile based on stored prescription data relative to a medication prescription prescribed to one or more patients; determining, by the one or more processors of the first computing system based on the prescription profile, whether or not the medication prescription is eligible to be covered by an insurance plan of each of the one or more patients; and when the medication prescription is determined to not be eligible to be covered by at least one of the insurance plans of the one or more patients or is determined to be eligible to be covered by at least one of the insurance plans of the one or more patients but the medication prescription is associated with a copay above a threshold cost relative to the at least one of the insurance plans: generating bidding quotes for the medication prescription by providing, via a network interface of the first computing system, the prescription profile of the stored prescription data to a plurality of pharmacy computing systems that are operated by a plurality of pharmacies; receiving, from one or more of the plurality of pharmacy computing systems via the network interface, the bidding quotes for the medication prescription at the first computing system, wherein each of the plurality of bidding quotes is generated by one of the plurality of pharmacy computing systems based on the prescription profile; sending, via the network interface of the first computing system, one or more notifications to each of the one or more patient computing systems for review by respective patients of the one or more patients of the one or more patient computing systems via respective user interfaces of the one or more patient computing systems; receiving, by the network interface of the first computing system, one or more notifications containing information relative to a selection of one of the bidding quotes for each of the one or more patients of the one or more patient computing systems; and sending, to respective pharmacy computing systems of the plurality of pharmacy computing systems associated with the one or more of the selected one of the bidding quotes via the network interface of the first computing system, one or more notifications containing information relative to the selection of the one of the bidding quotes.
Clause 8, the method of Clause 7, wherein the prescription provider is one of: a doctor, a pharmacist, a nurse practitioner, a physician assistant, or a clinical psychologist.
Clause 9, the method of Clause 7, wherein the prescription profile comprises a number of the one or more patients that are prescribed the medication prescription.
Clause 10, the method of Clause 7, wherein each of the one or more notifications containing the information relative to the selection of the one of the bidding quotes comprises identifying information of the one or more patients associated with each of the one or more notifications containing the information relative to the selection of the one of the bidding quotes.
Clause 11, the method of Clause 10, further comprising: sending, to the one or more patient computing systems of the plurality of patient computing systems associated with the one or more notifications containing the information relative to the selection of the one of the bidding quotes via the network interface of the first computing system, one or more notifications containing identifying information of the respective pharmacy computing systems of the plurality of pharmacy computing systems associated with the one or more of the selected one of the bidding quotes.
Clause 12, the method of Clause 7, wherein the generating of the bidding quotes for the medication prescription is performed at a scheduled interval, further wherein the scheduled interval is communicated to the system from a respective one of the one or more patients via the one or more patient computing systems.
Clause 13, a non-transitory computer-readable storage medium storing one or more programs configured to be executed by one or more processors of a first computing system having a network interface, the one or more programs including instructions for: generating, by the one or more processors of the first computing system, a prescription profile based upon stored prescription data relative to a medication prescription prescribed to one or more patients; determining, by the one or more processors of the first computing system based on the prescription profile, whether or not the medication prescription is eligible to be covered by an insurance plan of each of the one or more patients; and when the medication prescription is determined to not be eligible to be covered by at least one of the insurance plans of the one or more patients or is determined to be eligible to be covered by at least one of the insurance plans of the one or more patients but the medication prescription is associated with a copay above a threshold cost relative to the at least one of the insurance plans: generating bidding quotes for the medication prescription by providing, via the network interface of the first computing system, the prescription profile of the stored prescription data to a plurality of pharmacy computing systems that are operated by a plurality of pharmacies; receiving, from one or more of the plurality of pharmacy computing systems via the network interface, the bidding quotes for the medication prescription at the first computing system, wherein each of the plurality of bidding quotes is generated by one of the plurality of pharmacy computing systems based on the prescription profile; sending, via the network interface of the first computing system, one or more notifications to each of the one or more patient computing systems for review by respective patients of the one or more patients of the one or more patient computing systems via respective user interfaces of the one or more patient computing systems; receiving, by the network interface of the first computing system, one or more notifications containing information relative to a selection of one of the bidding quotes for each of the one or more patients of the one or more patient computing systems; and sending, to respective pharmacy computing systems of the plurality of pharmacy computing systems associated with the one or more of the selected one of the bidding quotes via the network interface of the first computing system, one or more notifications containing information relative to the selection of the one of the bidding quotes.
Clause 14, the non-transitory computer-readable storage medium of Clause 13, wherein the prescription provider is one of: a doctor, a pharmacist, a nurse practitioner, a physician assistant, or a clinical psychologist.
Clause 15, the non-transitory computer-readable storage medium of Clause 13, wherein the prescription profile comprises a number of the one or more patients that are prescribed the medication prescription.
Clause 16, the non-transitory computer-readable storage medium of Clause 13, wherein each of the one or more notifications containing the information relative to the selection of the one of the bidding quotes comprises identifying information of the one or more patients associated with each of the one or more notifications containing the information relative to the selection of the one of the bidding quotes.
Clause 17, the non-transitory computer-readable storage medium of Clause 16, the one or more programs further including instructions for: sending, to the one or more patient computing systems of the plurality of patient computing systems associated with the one or more notifications containing the information relative to the selection of the one of the bidding quotes, one or more notifications containing identifying information of the respective pharmacy computing systems of the plurality of pharmacy computing systems associated with the one or more of the selected one of the bidding quotes.
Clause 18, the non-transitory computer-readable storage medium of Clause 13, wherein the generating of the bidding quotes for the medication prescription is performed at a scheduled interval, further wherein the scheduled interval is communicated to the system from a respective one of the one or more patients via the one or more patient computing systems.

What is claimed is:

1. A system for providing a procurement of prescription medication quotes based on a medication prescription, comprising:
a network interface communicatively configured to couple the system to one or more patient computing systems of one or more patients, a prescription provider computing system, and a plurality of pharmacy computing systems;
one or more data storage devices configured to store prescription data relative to the medication prescription prescribed by a prescription provider to one or more patients; and
one or more tangible, non-transitory memories storing computer-executable instructions that, when executed by one or more processors of the system, cause the system to:
generate a prescription profile based upon the stored prescription data relative to the medication prescription prescribed to the one or more patients;
determine, based on the prescription profile, whether or not the medication prescription is eligible to be covered by an insurance plan of each of the one or more patients; and
when the medication prescription is determined to not be eligible to be covered by at least one of the insurance plans of the one or more patients or is determined to be eligible to be covered by at least one of the insurance plans of the one or more patients but the medication prescription is associated with a copay above a threshold cost relative to the at least one of the insurance plans:
generate bidding quotes for the medication prescription by providing, via the network interface, the prescription profile of the stored prescription data to the plurality of pharmacy computing systems;
receive, from one or more of the plurality of pharmacy computing systems via the network interface, the bidding quotes for the medication prescription at the system, wherein each of the plurality of bidding quotes is generated by one of the plurality of pharmacy computing systems based on the prescription profile;
send one or more notifications to each of the one or more patient computing systems for review by respective patients of the one or more patients of the one or more patient computing systems via respective user interfaces of the one or more patient computing systems;
receive, by the system, one or more notifications containing information relative to a selection of one of the bidding quotes for each of the one or more patients of the one or more patient computing systems; and
send, to respective pharmacy computing systems of the plurality of pharmacy computing systems associated with the one or more of the selected one of the bidding quotes via the network interface, one or more notifications containing information relative to the selection of the one of the bidding quotes.

2. The system of claim 1, wherein the prescription provider is one of: a doctor, a pharmacist, a nurse practitioner, a physician assistant, or a clinical psychologist.

3. The system of claim 1, wherein the prescription profile comprises a number of the one or more patients that are prescribed the medication prescription.

4. The system of claim 1, wherein each of the one or more notifications containing the information relative to the selection of the one of the bidding quotes comprises identifying information of the one or more patients associated with each of the one or more notifications containing the information relative to the selection of the one of the bidding quotes.

5. The system of claim 4, wherein the computer-executable instructions are further executable to:
send, to the one or more patient computing systems of the plurality of patient computing systems associated with the one or more notifications containing the information relative to the selection of the one of the bidding quotes, one or more notifications containing identifying information of the respective pharmacy computing systems of the plurality of pharmacy computing systems associated with the one or more of the selected one of the bidding quotes.

6. The system of claim 1, wherein the generating of the bidding quotes for the medication prescription is performed at a scheduled interval, further wherein the scheduled interval is communicated to the system from a respective one of the one or more patients via the one or more patient computing systems.

7. A computer-implemented method for providing a procurement of prescription medication quotes based on a medication prescription, comprising:
generating, by one or more processors of a first computing system, a prescription profile based on stored prescription data relative to a medication prescription prescribed to one or more patients;
determining, by the one or more processors of the first computing system based on the prescription profile, whether or not the medication prescription is eligible to be covered by an insurance plan of each of the one or more patients; and
when the medication prescription is determined to not be eligible to be covered by at least one of the insurance plans of the one or more patients or is determined to be eligible to be covered by at least one of the insurance plans of the one or more patients but the medication prescription is associated with a copay above a threshold cost relative to the at least one of the insurance plans:
generating bidding quotes for the medication prescription by providing, via a network interface of the first computing system, the prescription profile of the stored prescription data to a plurality of pharmacy computing systems that are operated by a plurality of pharmacies;
receiving, from one or more of the plurality of pharmacy computing systems via the network interface, the bidding quotes for the medication prescription at the first computing system, wherein each of the plurality of bidding quotes is generated by one of the plurality of pharmacy computing systems based on the prescription profile;
sending, via the network interface of the first computing system, one or more notifications to each of the one or more patient computing systems for review by respective patients of the one or more patients of the one or more patient computing systems via respective user interfaces of the one or more patient computing systems;
receiving, by the network interface of the first computing system, one or more notifications containing information relative to a selection of one of the bidding quotes for each of the one or more patients of the one or more patient computing systems; and
sending, to respective pharmacy computing systems of the plurality of pharmacy computing systems associated with the one or more of the selected one of the bidding quotes via the network interface of the first computing system, one or more notifications containing information relative to the selection of the one of the bidding quotes.

8. The method of claim 7, wherein the prescription provider is one of: a doctor, a pharmacist, a nurse practitioner, a physician assistant, or a clinical psychologist.

9. The method of claim 7, wherein the prescription profile comprises a number of the one or more patients that are prescribed the medication prescription.

10. The method of claim 7, wherein each of the one or more notifications containing the information relative to the selection of the one of the bidding quotes comprises identifying information of the one or more patients associated with each of the one or more notifications containing the information relative to the selection of the one of the bidding quotes.

11. The method of claim 10, further comprising: sending, to the one or more patient computing systems of the plurality of patient computing systems associated with the one or more notifications containing the information relative to the selection of the one of the bidding quotes via the network interface of the first computing system, one or more notifications containing identifying information of the respective pharmacy computing systems of the plurality of pharmacy computing systems associated with the one or more of the selected one of the bidding quotes.

12. The method of claim 7, wherein the generating of the bidding quotes for the medication prescription is performed at a scheduled interval, further wherein the scheduled interval is communicated to the system from a respective one of the one or more patients via the one or more patient computing systems.

13. A non-transitory computer-readable storage medium storing one or more programs configured to be executed by one or more processors of a first computing system having a network interface, the one or more programs including instructions for:
generating, by the one or more processors of the first computing system, a prescription profile based upon stored prescription data relative to a medication prescription prescribed to one or more patients;
determining, by the one or more processors of the first computing system based on the prescription profile, whether or not the medication prescription is eligible to be covered by an insurance plan of each of the one or more patients; and
when the medication prescription is determined to not be eligible to be covered by at least one of the insurance plans of the one or more patients or is determined to be eligible to be covered by at least one of the insurance plans of the one or more patients but the medication prescription is associated with a copay above a threshold cost relative to the at least one of the insurance plans:
generating bidding quotes for the medication prescription by providing, via the network interface of the first computing system, the prescription profile of the stored prescription data to a plurality of pharmacy computing systems that are operated by a plurality of pharmacies;
receiving, from one or more of the plurality of pharmacy computing systems via the network interface, the bidding quotes for the medication prescription at the first computing system, wherein each of the plurality of bidding quotes is generated by one of the plurality of pharmacy computing systems based on the prescription profile;
sending, via the network interface of the first computing system, one or more notifications to each of the one or more patient computing systems for review by respective patients of the one or more patients of the one or more patient computing systems via respective user interfaces of the one or more patient computing systems;
receiving, by the network interface of the first computing system, one or more notifications containing information relative to a selection of one of the bidding quotes for each of the one or more patients of the one or more patient computing systems; and
sending, to respective pharmacy computing systems of the plurality of pharmacy computing systems associated with the one or more of the selected one of the bidding quotes via the network interface of the first computing system, one or more notifications containing information relative to the selection of the one of the bidding quotes.

14. The non-transitory computer-readable storage medium of claim 13, wherein the prescription provider is one of: a doctor, a pharmacist, a nurse practitioner, a physician assistant, or a clinical psychologist.

15. The non-transitory computer-readable storage medium of claim 13, wherein the prescription profile comprises a number of the one or more patients that are prescribed the medication prescription.

16. The non-transitory computer-readable storage medium of claim 13, wherein each of the one or more notifications containing the information relative to the selection of the one of the bidding quotes comprises identifying information of the one or more patients associated with each of the one or more notifications containing the information relative to the selection of the one of the bidding quotes.

17. The non-transitory computer-readable storage medium of claim 16, the one or more programs further including instructions for:
sending, to the one or more patient computing systems of the plurality of patient computing systems associated with the one or more notifications containing the information relative to the selection of the one of the bidding quotes, one or more notifications containing identifying information of the respective pharmacy computing systems of the plurality of pharmacy computing systems associated with the one or more of the selected one of the bidding quotes.

18. The non-transitory computer-readable storage medium of claim 13, wherein the generating of the bidding quotes for the medication prescription is performed at a scheduled interval, further wherein the scheduled interval is communicated to the system from a respective one of the one or more patients via the one or more patient computing systems.
